(19) United States
(12) Patent Application Publication
(10) Pub. No.: US 2015/0343147 A1
(43) Pub. Date: Dec. 3, 2015

(54) INJECTION DEVICE

(71) Applicant: Allergan, Inc., Irvine, CA (US)

(72) Inventors: Ethan Franklin, Goleta, CA (US); Zachary Dominguez, Santa Barbara, CA (US)

(21) Appl. No.: 14/723,127

(22) Filed: May 27, 2015

Related U.S. Application Data
(60) Provisional application No. 62/004,617, filed on May 29, 2014.

(51) Int. Cl. A61M 5/315

(52) U.S. Cl. CPC A61M 5/31511; A61M 5/31576; A61M 5/31586; A61M 2005/3152; A61M 2005/3139

(57) ABSTRACT

Disclosed herein are devices and methods that can provide assistance with extruding and/or aspirating high viscosity materials such as dermal fillers.

### CROSS-REFERENCE TO RELATED APPLICATIONS

This application claims priority to, and the benefit of, U.S. Provisional Patent Application No. 62/004,617, filed on May 29, 2014, the entire disclosure of which is incorporated herein by this specific reference.

### BACKGROUND

Injection of liquids, gels, and gases through a syringe is common practice in many applications including both medical and nonmedical purposes. When injecting highly viscous materials requiring high force and/or pressure using a standard syringe, users may experience a high injection force for extrusion and/or aspiration. Some medical examples of high force applications are fat grafting and dermal fillers, for example, injectable, hyaluronic acid-based dermal fillers for reducing the appearance of wrinkles.

Many of these injectable materials, for example, dermal fillers and fat grafting materials, are not easily extruded through standard syringes and accompanying cannula. These materials tend to provide significant resistance when pushed through a narrow cannula. The problem is even more exacerbated by the fact that these materials are often used for detailed precision work in facial contouring and body sculpting.

A need exists for devices that can be attached to or used in place of a standard syringe that provide better control over dosing of relatively difficult to inject materials, for example, dermal fillers, fat grafting materials and the like.

### SUMMARY

Devices and methods, in particular syringes, are described herein that can provide assistance with extruding and/or aspirating high viscosity materials such as, but not limited to, gels or fluids. These viscous materials can be, for example, but not limited to, a dermal filler, a fat grafting material, an epoxy, caulking, or a combination thereof. The devices can be a variation of a standard syringe and can include electromechanical or mechanical assistance with injection or extrusion.

Note that in standard syringes the force applied to the plunger is the same force that is delivered to the piston. Described herein are embodiments of assisted syringes wherein the force applied to the plunger can be different than the force applied to the piston.

Described herein are mechanically assisted syringes having a clutch bearing drive comprising: a plunger at a proximal end of the syringe, the plunger configured to receive a first force applied by a user in a first lateral direction to achieve a first displacement distance; a piston at a distal end of the syringe configured to move in a second lateral direction a second displacement distance; at least one gear reduction configured to receive the plunger and transfer the movement of the plunger to the piston and translate the first distance of the plunger into the second distance of the piston; at least one clutch bearing integrated into the at least one gear reduction such that the at least one gear reduction is engaged when the plunger is moved the first lateral direction, but the at least one gear reduction is disengaged when the plunger is moved in a lateral direction opposite to the first lateral direction; a toggle switch configured to reverse the orientation of the at least one clutch bearing; wherein the first displacement distance of the plunger is less than the second displacement distance of the piston.

Further described herein is a method of assisting an injection of a viscous material, the method comprising: providing a mechanical assisted syringe having a clutch bearing drive comprising: a plunger at a proximal end of the syringe, the plunger configured to receive a first force applied by a user in a first lateral direction to achieve a first displacement distance; a piston at a distal end of the syringe configured to move in a second lateral direction a second displacement distance; at least one gear reduction configured to receive the plunger and transfer the movement of the plunger to the piston and translate the first distance of the plunger into the second distance of the piston; at least one clutch bearing integrated into the at least one gear reduction such that the at least one gear reduction is engaged when the plunger is moved the first lateral direction, but the at least one gear reduction is disengaged when the plunger is moved in a lateral direction opposite to the first lateral direction; a toggle switch configured to reverse the orientation of the at least one clutch bearing; wherein the first displacement distance of the plunger is less than the second displacement distance of the piston.

### BRIEF DESCRIPTION OF THE DRAWINGS

Embodiments of the present description are illustrated by way of example, and not by way of limitation, in the figures of the accompanying drawings and in which like reference numerals refer to similar elements, wherein:

FIG. 1 Illustrates a perspective view of a 3D depiction of one embodiment.

FIG. 2 Is a top view of one embodiment.

FIG. 3 Illustrates a lateral view highlighting same axis functionality of plunger and piston.

### DETAILED DESCRIPTION

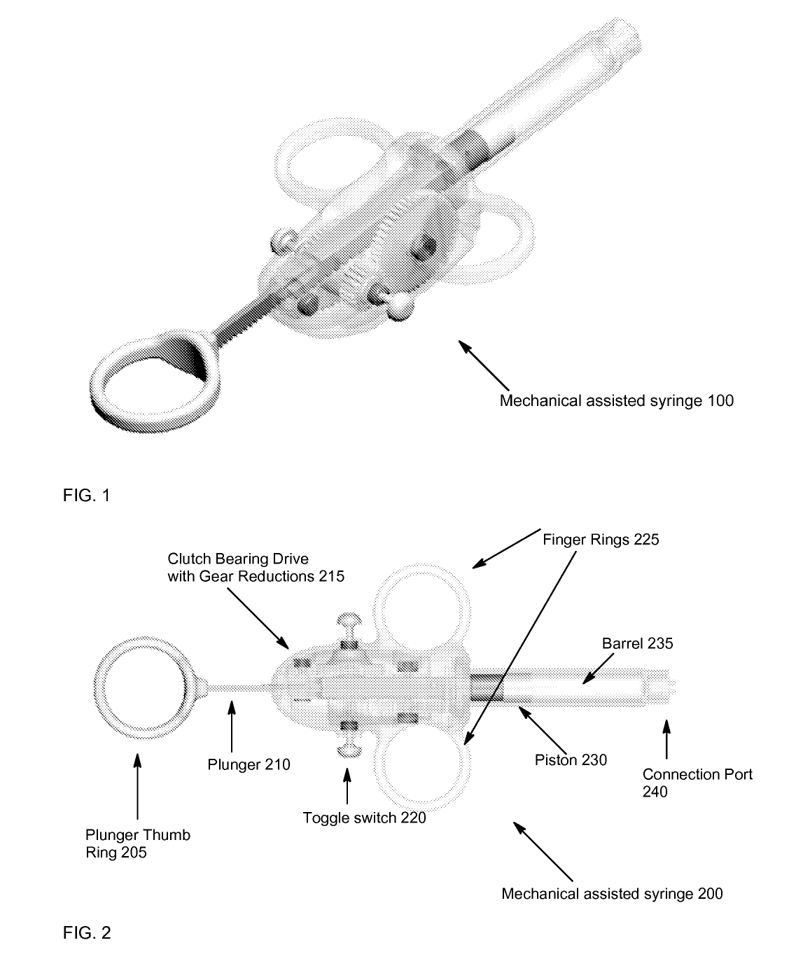

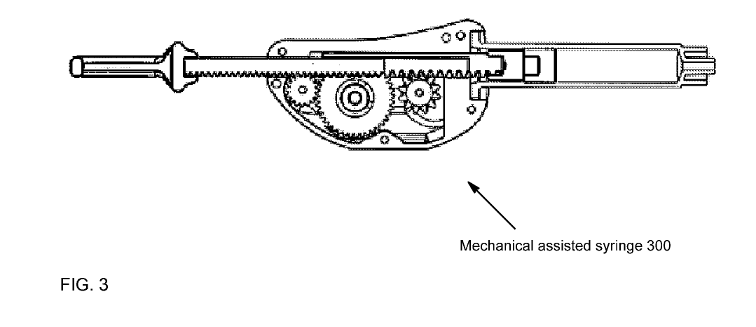

Generally described herein are devices and methods for extruding and/or aspirating high viscosity materials. In some embodiments, these high viscosity materials can be gels or fluids. The devices can be a variation of a standard syringe including mechanical or electro-mechanical elements, while keeping a familiar, syringe-style user interface. The embodiments can be applied to either reusable or single use applications. In some embodiments, the syringes described can be completely independent from a standard syringe (see FIGS. 1 and 2).

The syringes described herein can be assisted syringes. The assisted syringes described herein can achieve at least one of: relieving the user from high injection/aspiration forces associated with highly viscous gels/fluids and/or high gauge needles; having little impact on the user's preferred style of injection; and keeping extrusion/aspiration speed to a controlled level. The assisted syringes can provide assistance via gear reductions, which allow in increase in force applied to extrusion or aspiration proportional to the increase in displacement.

In general, the assisted syringes described herein can include a variety of components found in standard syringes such as, but not limited to, a plunger located at a proximal end, a piston located at a distal end, an internal volume (e.g., barrel) to hold a material to be injected or to receive a material being aspirated, and a connection port, or other connection type, examples including without limitation: luer tip or other connection device or a needle or other cannula permanently attached (see FIG. 2). In some embodiments, the plunger located at the proximal end is configured to receive a first force applied by a user in a first lateral direction to achieve a first displacement distance. In some embodiments, the piston at the distal end of the syringe is configured to move in a second lateral direction a second displacement distance. In some embodiments, the first displacement distance of the plunger is less than the second displacement distance.

As known in the art, the relationship between work (W), force (F), and distance (D), are shown in the following formula.

$$W = F * D$$

$$and so , \\ F = \frac{W}{D}$$

This equation shows that as you increase the distance traveled you can decrease the amount of force needed to perform the same amount of work. In other words, the force applied by the thumb to the plunger will increase the force applied to the piston proportional to the increase in distance traveled by the plunger. The gear reductions in the device could be increased to allow for the extrusion of products of higher viscosity, or decreased for products of lower viscosity to minimize the required plunger displacement for extrusion of the product.

In one embodiment, as illustrated in FIG. 2, mechanical syringe 200 can be provided to reduce injection force. Plunger 210 can be actuated by a user's thumb via plunger thumb ring 205. The user can hold the mechanical syringe with a thumb in the thumb ring and one finger in each of two finger rings 225. The user then actuates the plunger which then propagates force through a clutch bearing drive with gear reductions 215. The drive can include one or more gear reductions dependent on the amplification of force required based on the properties of the material being extruded or aspirated. Clutch bearings also included in the drive allow the drive train to be reset during operation allowing the reset of the plunger independently of piston 230. Toggle switch 220 can be activated to either allow only forward or backward movement of the piston, resulting in extrusion or aspiration of product respectively. In some embodiments, the toggle switch can be designed to cause extrusion on backward movement of the plunger or aspiration on foreword movement of the plunger. The force transferred through the drive acts on the piston to either extrude or aspirate product depending on its change in position. Forward motion of the piston would cause extrusion of product contained in barrel 235 out connection port 240, and reverse movement would cause aspiration of product into barrel 235, via connection port 240.

One or more clutch bearings can be integrated into the gear reductions such that the transmission is engaged in one direction and disengaged in the opposite direction. This clutch bearing is useful in that it allows the drive train to be reset repeatedly during an injection. In other words once the thumb has traveled the allowable distance, a clutch bearing would allow the plunger to be reset or pulled back independently of the piston of the assisted syringe device and thereby allowing the continuing of applied pressure to continue the function of the injecting device. In some embodiments, this would allow continued application of a viscous dermal filler. In some embodiments, at least one clutch bearing integrated into the at least one gear reduction such that the at least one gear reduction is engaged when the plunger is moved the first lateral direction, but the at least one gear reduction is disengaged when the plunger is moved in a lateral direction opposite to the first lateral direction. In some embodiments, at least one gear reduction can be configured to receive the plunger, transfer the movement of the plunger to the piston, and translate the first distance of the plunger into the second distance of the piston.

In some embodiments, the clutch bearing device used is a drawn-cup roller clutch, or one-way locking needle roller bearing. In other embodiments, the clutch bearing device could instead be a ratchet or other one-way mechanism. Clutch bearings might be useful as they are relatively compact mechanisms and have minimal backlash. This would allow the overall embodiment to maintain a compact form factor as well as minimizing “slop” or “looseness” at the input rack of the device. However, a ratchet or other one way mechanism might prove advantageous from a cost perspective.

Some embodiments may feature a concentric rack design. Note that both the initial rack (driven by the plunger) and the final rack (that drives the piston) act along the same axis (see FIGS. 1 and 2). This can be achieved by cutting out a groove in the final rack through which the initial rack can travel. This feature provides the user a similar feel in hand compared to injection with a standard syringe. Alternative embodiments that do not incorporate this feature tend to feel unnatural because the two lines of action are not concentric.

In some embodiments, the assisted syringe device can include a toggle switch, which would allow the device to switch between forward and reverse action. In some embodiments, the toggle switch mechanism switches between two roller clutches that are oriented in opposite directions. The result is the ability to drive the piston forward for extrusion or pull it backward for aspiration. In some embodiments, this could allow forward motion of the plunger to cause the piston to pull backward allowing aspiration. In some embodiments, this switch would lockout the reverse action (to allow for one way movement of the piston for example) and the activation of the switch would allow reverse action on the plunger to cause aspiration by the piston. In some embodiments, the toggle switch could allow reverse action of applied pressure to cause extrusion. In some embodiments, the toggle switch could allow forward action of applied pressure to cause extrusion. In some embodiments a toggle switch can be configured to reverse the orientation of the at least one clutch bearing.

The syringes described herein can provide a high force to extrude a viscous material. This stronger extrusion force applied by the piston can be based on a lesser force applied to the syringe's plunger. The extrusion force can be greater than about 1 Newton (N), greater than about 2 N, greater than about 3 N, greater than about 4 N, greater than about 5 N, greater than about 10 N, greater than about 20 N, greater than about 40 N, greater than about 60 N, greater than about 80 N, greater than about 100 N, from about 1 N to about 20 N, about 1 N to about 100 N, or about 20 N to about 100 N, about 10 N to about 70 N, about 60 N to about 140 N, or about 19 N, or about 26 N or about 56 N, or about 80 N, or about 120 N, or any force in a range bounded by, or between, any of these values.

The syringes described herein can further provide a high force to aspirate a viscous material. This stronger aspiration force resulting from the backward movement of the piston can be based on a lesser force applied to the syringe's plunger. The aspiration force can be greater than about 1 N, greater than about 2 N, greater than about 3 N, greater than about 4 N, greater than about 5 N, greater than about 10 N, greater than about 20 N, greater than about 40 N, greater than about 60 N, greater than about 80 N, greater than about 100 N, from about 1 N to about 20 N, about 1 N to about 100 N, or about 20 N to about 100 N, about 10 N to about 70 N, about 60 N to about 140 N, or about 19 N, or about 26 N or about 56 N, or about 80 N, or about 120 N, or any force in a range bounded by, or between, any of these values.

In some embodiments, the devices can further include electromechanics for pressure relief and/or adding force and/or a unit to control and sense forward and reverse functions of the devices.

In some embodiments, devices can be motorized.

The assisted injecting device can inject highly viscous materials through a various range of needle gauges. In some embodiments, the needle can have a gauge as high as 50, in the range of about 10 to about 33, or about 30. The devices can extrude material at a rate of about 0.001 to about 1 mL/sec. Other extrusion speeds can be between 0.004 to 0.05 mL/sec. The rate of extrusion may be dependent on the viscosity of the material being extruded and the density of the tissue or material being injected. A highly viscous material may require much more extrusion force than will a low viscosity material. A person skilled in the art can determine the rate of injection relative to the viscosity of the material and density of the tissue or material being injected.

In some embodiments, the materials to be extruded may be non-Newtonian fluids or mixtures of Newtonian and non-Newtonian fluids. Such fluids can have inconsistent and/or unpredictable force-to move requirements. Such fluids can have high yield points requiring high stall torque requirements. Non-Newtonian fluids may have high yield points but have rapid drops in force-to-move requirements after the yield point is overcome. As such, the syringes described herein can accommodate for rapid changes in extrusion force requirements.

Some embodiments can include, a variable slider that can allow for dynamic injection speeds, and not just a binary switch based operation. A variable slider can ramp up the extrusion speed depending on the amount of force applied to the plunger. Likewise, a variable slider can ramp up the aspiration speed depending on the amount of backward force applied to the plunger.

The assistance devices described herein can be relatively simple to use, with a minimal learning curve. The user can fill their syringe using any standard means or can use an assisted method or device as described. Then, injection can be made easier via the assistance provided by the devices described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A mechanically assisted syringe having a clutch bearing drive, the syringe comprising:
a plunger at a proximal end of the syringe, the plunger configured to receive a first force applied by a user in a first lateral direction to achieve a first displacement distance;
a piston at a distal end of the syringe configured to move in a second lateral direction a second displacement distance;
at least one gear reduction configured to receive the plunger and transfer the movement of the plunger to the piston and translate the first distance of the plunger into the second distance of the piston;
at least one clutch bearing integrated into the at least one gear reduction such that the at least one gear reduction is engaged when the plunger is moved the first lateral direction, but the at least one gear reduction is disengaged when the plunger is moved in a lateral direction opposite to the first lateral direction;
a toggle switch configured to reverse the orientation of the at least one clutch bearing;
wherein the first displacement distance of the plunger is less than the second displacement distance of the piston.

2. The mechanical syringe of claim 1, wherein the clutch bearing allows the drive train to be reset repeatedly during an injection.

3. The mechanical syringe of claim 1, wherein the piston causes extrusion or aspiration of product.

4. The mechanical syringe of claim 1, wherein the clutch bearing is a drawn cup roller clutch.

5. The mechanical syringe of claim 1, wherein the clutch bearing is a one way locking needle roller bearing clutch.

6. The mechanical syringe of claim 1, wherein the clutch is a ratcheting one way mechanism.

7. The mechanical syringe of claim 1, wherein first lateral direction and the second lateral direction are coaxial.

8. The mechanical syringe of claim 1, wherein the toggle switch allows the device to switch between forward and reverse action.

9. The mechanical syringe of claim 8, wherein the forward action results in extrusion.

10. The mechanical syringe of claim 8, wherein the forward action results in aspiration.

11. The mechanical syringe of claim 8, wherein the reverse action results in aspiration.

12. The mechanical syringe of claim 8, wherein the reverse action results in extrusion.

13. A method of assisting an injection of a viscous material, the method comprising:
providing a mechanically assisted syringe, the syringe comprising:
a plunger at a proximal end of the syringe, the plunger configured to receive a first force applied by a user in a first lateral direction to achieve a first displacement distance;
a piston at a distal end of the syringe configured to move in a second lateral direction a second displacement distance;
at least one gear reduction configured to receive the plunger and transfer the movement of the plunger to the piston and translate the first distance of the plunger into the second distance of the piston;
at least one clutch bearing integrated into the at least one gear reduction such that the at least one gear reduction is engaged when the plunger is moved the first lateral direction, but the at least one gear reduction is disengaged when the plunger is moved in a lateral direction opposite to the first lateral direction;
a toggle switch configured to reverse the orientation of the at least one clutch bearing;
wherein the first displacement distance of the plunger is less than the second displacement distance of the piston; and
applying a force to the plunger.

14. The method of claim 13, wherein the assisted syringe is the mechanical assisted syringe of claim 1.
